(19) United States
(12) Patent Application Publication
(10) Pub. No.: US 2021/0236230 A1
(43) Pub. Date: Aug. 5, 2021

(54) PROTECTIVE COVER FOR BODY PART FIXATION DEVICE

(71) Applicant: Medtec, Inc., Orange City, IA (US)

(72) Inventors: Craig Copple, Coralville, IA (US); Gregory Nephi Nordgren, North Liberty, IA (US); Roger F. Wilson, Sarasota, FL (US)

(21) Appl. No.: 17/049,067

(22) PCT Filed: Apr. 19, 2019
(86) PCT No.: PCT/US2019/028261

§ 371 Date: Oct. 20, 2020

Related U.S. Application Data
(60) Provisional application No. 62/665,136, filed on May 1, 2018.

(51) Int. Cl. A61B 90/18; B29C 51/42; A61B 90/40

(52) U.S. Cl. CPC A61B 90/18; A61B 2090/401; A61B 90/40; B29C 51/424

(57) ABSTRACT

An assembly for a thermoplastic preform for securing body part is provided. The thermoplastic preform is deformable when heated to a softening temperature by an external heating source and hardens when cooled below the softening temperature. The assembly includes the thermoplastic preform and a protective cover surrounding the thermoplastic preform. The thermoplastic preform is sealed within the protective cover. The protective cover is heat resistant up to a temperature that exceeds the softening temperature for the thermoplastic preform. The protective cover transfers heat from the external heating source to the thermoplastic preform and prevents airborne and waterborne bacteria from contacting the thermoplastic preform.

### CROSS-REFERENCE TO RELATED APPLICATION

This application claims the benefit under 35 U.S.C. § 371 of International Application No. PCT/US2019/028261, filed Apr. 19, 2019, which claims priority under 35 U.S.C. § 119 based on U.S. Provisional Application No. 62/665,136 filed May 1, 2018, the disclosures of which are both hereby incorporated by reference herein.

### BACKGROUND

Certain types of medical treatments require that a portion of a human body be held in a same position to facilitate performance of the medical treatment upon that portion of the body. For example, when brain cancer patients undergo radiation treatment, their heads must be maintained in a precise, repeatable location for the treatment such that the underlying position of the brain tumor is fixed in space for the duration of the radiation treatment or treatments. Various different techniques have been used in the field of radiation oncology for holding body parts in a fixed position. In one such technique, a thermoplastic sheet of material is heated in an oven or water bath and stretched over the body part of the patient. Once cooled, the sheet hardens and is used on subsequent visits by the patient when getting treatment.

### BRIEF DESCRIPTION OF THE DRAWINGS

FIG. 1 is a front perspective view of a thermoplastic preform assembly including a thermoplastic mask within a vacuum sealed protective cover, according to an implementation described herein;

FIG. 2A is a front view of a frameless thermoplastic preform;

FIG. 2B is a front perspective view of a thermoplastic preform including a mounting frame, according to an implementation;

FIG. 2C is a rear perspective view of a thermoplastic preform including a mounting frame, according to another implementation;

FIG. 3 is a schematic front view of the protective cover of FIG. 1, according to an implementation;

FIG. 4A is an assembly view of the thermoplastic preform assembly of FIG. 1 according to an embodiment;

FIG. 4B is an assembly view of the thermoplastic preform assembly of FIG. 1 according to another embodiment;

FIG. 5 is a schematic illustrating placement of the assembly of FIG. 1 into a heating source, according to an implementation;

FIGS. 6, 7A, and 7B are flow diagrams of an exemplary process for using a thermoplastic preform assembly, according to an implementation described herein;

FIG. 8 is a schematic illustrating a thermoplastic preform formed over a patient;

FIGS. 9A and 9B are front perspective views of a thermoplastic preform assembly including a thermoplastic mask within a protective cover, according to another implementation described herein; and

FIG. 10A is an assembly view of the thermoplastic preform assembly of FIG. 9B; and

FIG. 10B is a schematic illustrating placement of the assembly of FIG. 9B into a heating source, according to an implementation.

### DETAILED DESCRIPTION OF THE PREFERRED EMBODIMENTS

The following detailed description refers to the accompanying drawings. The same reference numbers in different drawings may identify the same or similar elements. The following detailed description does not limit the invention.

A technique, in the field of radiation oncology, for holding body parts in a fixed position uses heat-formable structures (referred to as “body part fixation devices,” “thermoplastic retention device preforms,” or “thermoplastic preforms”) that include a sheet of retention material that is stretched over the body part of a patient. For example, for performing radiation treatment of a brain tumor, the heat-formable structure includes a mask having a sheet of retention material that is stretched over the patient's face. To form the mask over the patient's face, a hot water bath or dry oven may be first used to heat the material of the heat-formable structure such that the sheet of material becomes pliable and deformable. The heat-formable mask is then stretched over the patient's face, and the mask is allowed to cool and harden, permanently forming the mask to the shape of the face of the patient. Upon cooling, the mask, formed to the patient's face, creates a structure that can be used to hold the patient's head in a fixed position during radiation treatments. After the sheet of thermoplastic retention material of the mask is stretched over the body part of the patient, a frame portion of the mask is attached to a patient support table, or other structure, using an attachment mechanism(s).

The thermoplastic preforms are typically provided to customers in a box stacked on each other without separators or covers. The thermoplastic preforms are removed from the box when needed and placed in a heating source (e.g., either a water bath or dry oven) to be heated for an appropriate time and to a softening temperature. However, there is typically an issue with the sanitary conditions of the heating source. Water baths and dry ovens both generate bacteria because they are warm (e.g., about 60°-75° Celsius), but are not hot enough to kill bacteria at a boiling temperature. Such bacteria may attach to the thermoplastic preform that goes over the body part of the patient, causing possible unsanitary conditions for the patient.

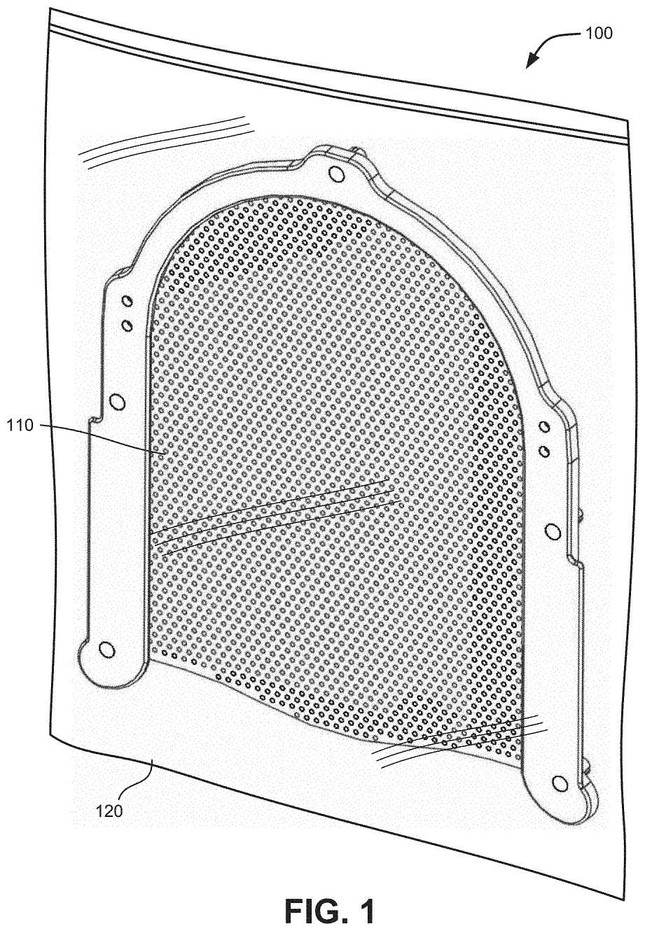

Exemplary embodiments described herein relate to improvements for limiting body part fixation devices to bacterial exposure. FIG. 1 is an illustration of a thermoplastic preform assembly 100 including a thermoplastic preform 110 within a protective cover 120, according to an implementation described herein. Protective cover 120 is applied over the thermoplastic preform 110 (shown herein as a mask for simplicity, but other preform shapes may be used) to protect the patient from possible exposure to unwanted bacteria which may attach during the process of heating thermoplastic preform 110 in either a water bath or dry oven. According to an implementation, protective cover 120 is vacuum sealed over thermoplastic preform 110 to remove unwanted air inside the protective covering. According to another implementation, protective cover 120 adheres to the surface of thermoplastic preform 110 to minimize air entrapment within the protective covering.

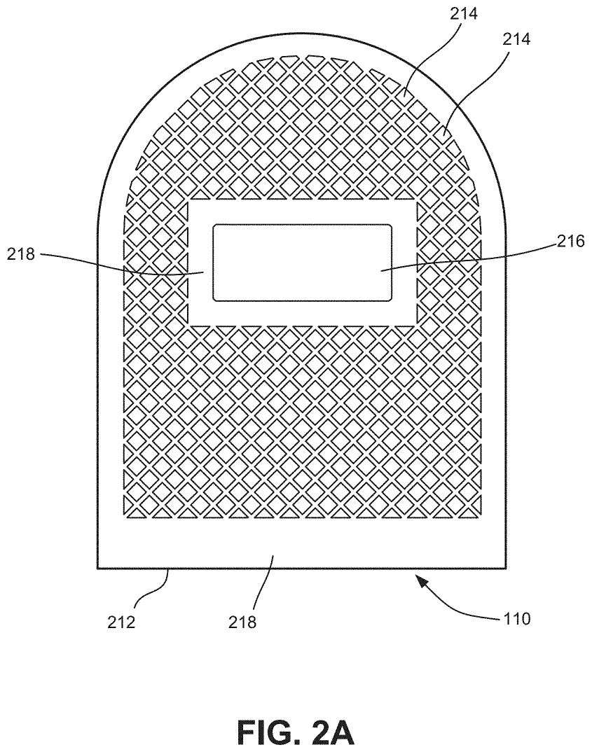

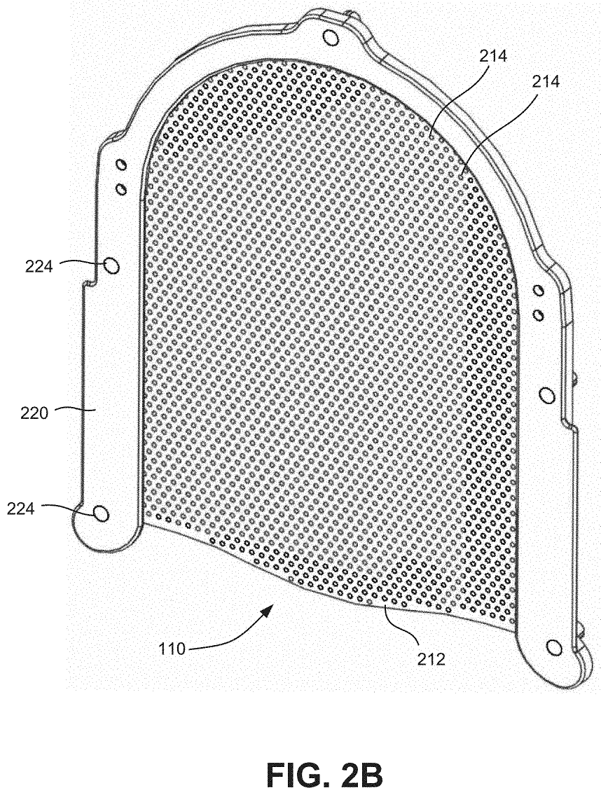

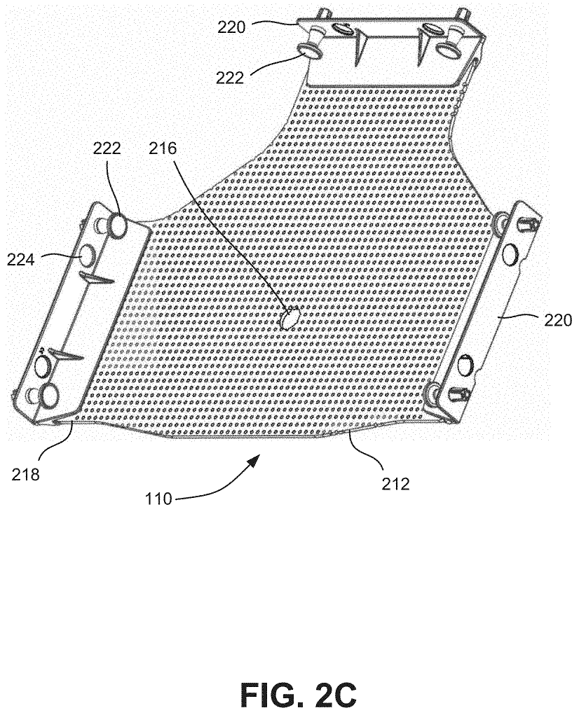

FIG. 2A is a front view of a thermoplastic preform 110, according to an implementation. FIG. 2B is a front perspective view of a thermoplastic preform 110 mounted in a frame 220, according to another implementation. FIG. 2C is a rear perspective view of a thermoplastic preform 110 mounted in a frame 220, according to another implementation. Referring to FIGS. 2A-2C, thermoplastic preforms preferably include different patterns which may provide advantages to medical technicians and patients. The patterns include an overall shape (e.g., a head shape, a head/neck/shoulders shape, a hip/pelvis shape, a torso shape, etc.) and also areas of different pattern density/strength may be needed to provide structural integrity while minimizing weight. In some cases, different hole or perforation patterns are used within a single preform. The holes or perforations may be added, for example, in low stress areas to reduce the overall weight of a preform/mask and to provide the impression of a less confining environment for a patient. As used herein, the term “thermoplastic” refers to heat-moldable thermoplastic materials, including medical-grade thermoplastic materials.

As shown in FIG. 2A, thermoplastic preform 110 may correspond to a face mask. Thermoplastic preform 110 may include a generally flat base layer 212 of thermoplastic material with holes 214 extending through a thickness of base layer 212. In one implementation, base layer 212 may include one or more solid portions 218 to provide reinforcement and/or a surface for attachment to a frame or mounting platform. Thermoplastic preform 110 may also include an opening 216 with an additional solid portion 218. Opening 216 may correspond, for example, to the location of a patient's eyes and/or nose. FIG. 2A provides an illustration of one of numerous variations of hole or perforation patterns (i.e., square-shaped holes) that may be used in base layer 212 for thermoplastic preform 110.

Base layer 212 may be made from a base polymer of polycaprolactone (PCL) and may be treated with chemicals to enhance the crosslinking of the polymer chains within the polymer when base layer 212 is exposed to radiation. After forming the base layer 212, radiation is applied to base layer 212. The radiated polymer blend creates a “memory” in the material that allows the material to retain a shape when cool. When the material is later heated to a softening temperature (e.g., about 55° C. to 75° C.), the preform can be shaped (e.g., around a body part of patient) without losing integrity and will maintain its shape once cooled.

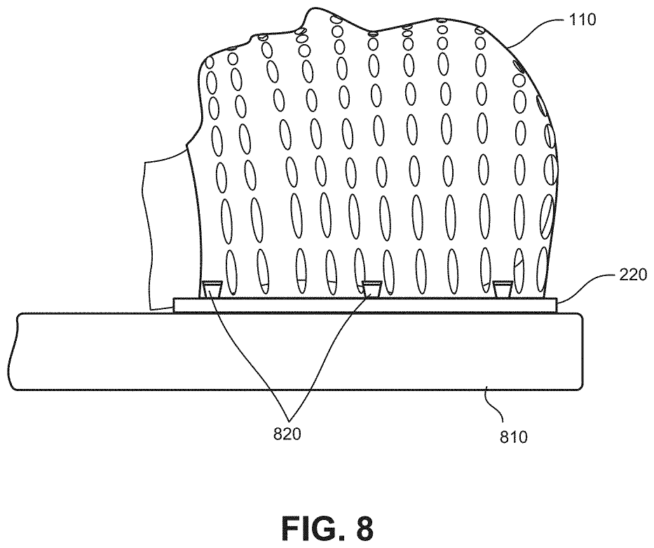

Referring to FIGS. 2B and 2C, frame 220 may be included with thermoplastic preform 110. Frame 220 may include a relatively rigid structure that may be mechanically fastened, glued, or otherwise secured to base layer 212. Frame 220 may be a single- or multi-piece structure that holds portions of thermoplastic preform 110 (e.g., solid portions 218), for example. Frame 220 may include mounting holes 224 to enable frame 220 to be secured to a mounting platform, table or another surface (e.g., after thermoplastic preform 110 has been placed and/or formed over a patient's head). As shown in FIG. 2B, pins (e.g., shown in FIG. 8 as pins 820, and supplied separately from assembly 100) may be inserted through mounting holes 224 to secure thermoplastic preform 110 in place. In other implementations, frame 220 may include integrated spring clips, pins, or other fastening mechanisms. For example, as shown in FIG. 2C, clips 222 may be integrated with frame 220 and included with assembly 100. In one implementation, frame 220 may be made of a different material than thermoplastic preform 110. Particularly, frame 220 may be made of a material that will not deform at temperatures used to soften base layer 212. Thus, frame 220 may provide support for base layer 212 during removal of thermoplastic preform 110 from protective cover 120 (e.g., after heating/softening). Furthermore, the non-malleable frame 220 ensures the position of mounting holes 224 can be maintained for consistent alignment with a corresponding headrest or mounting platform for a patient (e.g., as shown in FIG. 8). While the configuration of FIGS. 2A, 2B, and 2C shows thermoplastic preform 110 as a face mask, in other implementations, preform shapes for other body parts or combinations of body parts may be used.

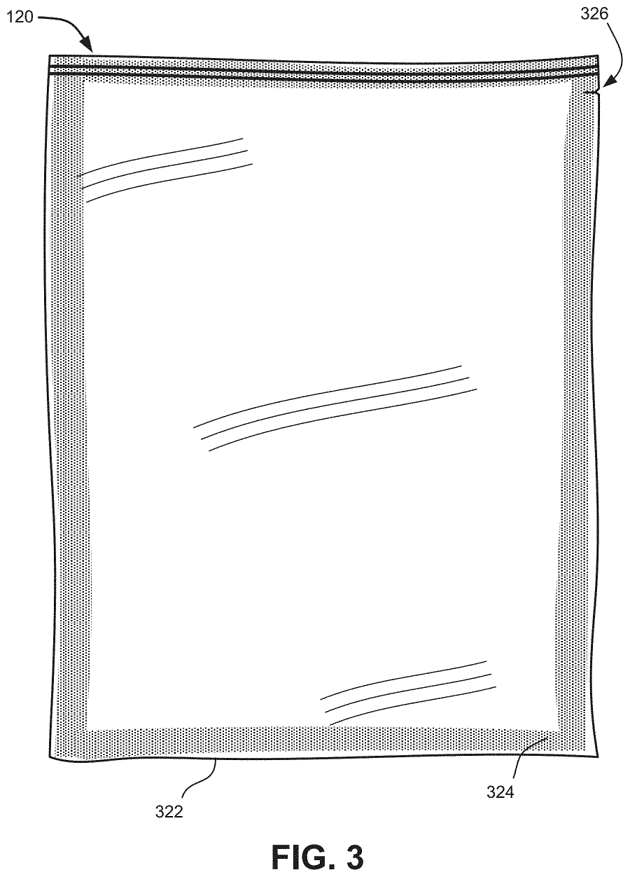

FIG. 3 is a schematic front view of protective cover 120. According to one implementation, protective cover 120 may include a bagging film 322 including an inner space from which air can be extracted. Bagging film 322 may be made of one or more layers of nylon, polyethylene, ethylene-vinyl alcohol, polyamide, polyethylene terephthalate, and/or another suitable material. In one implementation, as shown in FIG. 1, bagging film 322 may be substantially clear (e.g., transparent or slightly translucent) to permit thermoplastic preform 110 to be visible within protective cover 120. Bagging film 322 may include a high-temperature heat-stabilized film that can maintain a seal at temperatures above the softening temperature of thermoplastic preform 110. In another implementation, bagging film 322 may include oxygen and moisture barrier properties necessary to prevent thermoplastic preform 110 (e.g., when sealed within protective cover 120) from being exposed to bacteria in a water bath or dry oven.

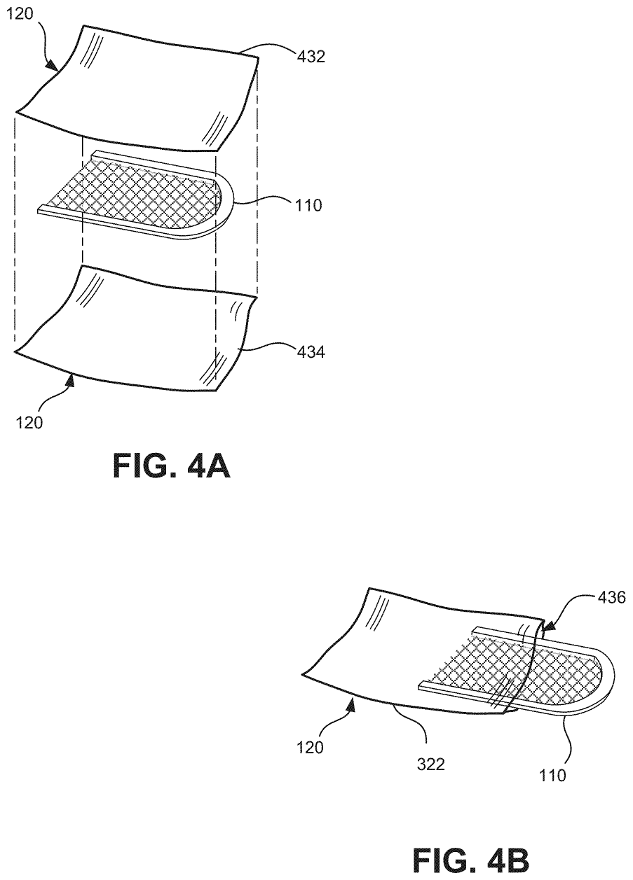

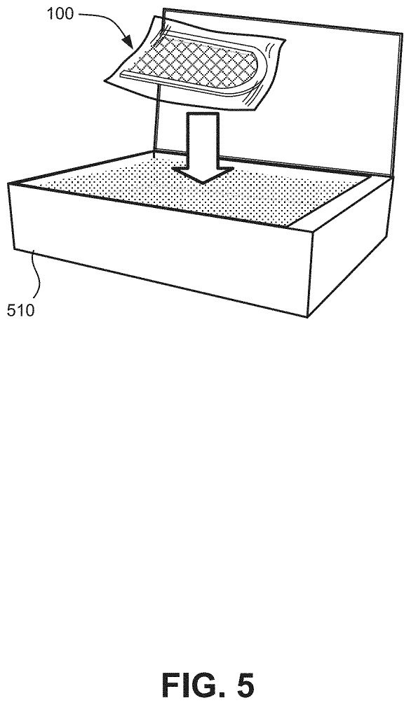

FIG. 4A is an assembly view of thermoplastic preform assembly 100 according to one embodiment. As shown in FIG. 4A, protective cover 120 may be formed over thermoplastic preform 110 using a top film sheet 432 and a bottom film sheet 434 of bagging film 322. Top film sheet 432 and bottom film sheet 434 may be made of the same or different materials. A chamber vacuum sealer (e.g., including an enclosed chamber where assembly 100 is sealed) may be used to seal seams along the perimeter of the adjoined top film sheet 432 and bottom film sheet 434 while creating a vacuum seal between top film sheet 432 and bottom film sheet 434.

FIG. 4B is an assembly view of the thermoplastic preform assembly 100 according to another embodiment. As shown in FIG. 4B, protective cover 120 may be supplied with bagging film 322 forming a pouch with an opening 436 into which thermoplastic preform 110 may be inserted. An external vacuum sealer may be used where a vacuum manifold (e.g., a small tube or snorkel) is inserted to remove air from inside protective cover 120 before sealing along opening 436.

As illustrated in FIG. 3, an adhesive 324 may be used to provide a continuous airtight seal between the top and bottom layers of bagging film 322 around the perimeter of thermoplastic preform 110. In another implementation, adhesive 324 may be used to seal the point (or port) where a vacuum manifold or port enters protective cover 120 to extract air. Adhesive 324 may include, for example, a flexible adhesive strip, mastic, epoxy, etc. In other implementations, a heat seal may be used, in place of, or in addition to, adhesive 324, to seal one or more seams of protective cover 120. Bagging film 322 also may be puncture resistant and heat resistant above the softening temperature of thermoplastic preform 110. In one implementation, bagging film 322 is heat resistant to at least 90° C.

In some implementations, protective cover 120 may use layers of other materials between the bagging film and thermoplastic preform 110. For example, a disposable release fabric may be used inside protective cover 120 to help channel air out of protective cover 120 during a suction/sealing process and to enable easier removal of thermoplastic preform 110 from protective cover 120. In other implementations, bagging film 322 may include a wrinkled type film or embossing that provides a texture designed to channel air and eliminate the need for breather fabric.

According to an implementation, protective cover 120 may include a notch 326 or pre-cut slit to provide easy opening by consumers. According to another implementation, protective cover 120, adhesive 324 may permit bagging film 322 (e.g., top film sheet 432 and bottom film sheet 434) to be manually pulled apart along one or more parts of the perimeter of protective cover 120.

Referring collectively to FIGS. 1-4B, assembly 100 may be formed by enclosing thermoplastic preform 110 within protective cover 120 as part of a manufacturing process (e.g., prior to delivery to a customer). After thermoplastic preform 110 is inserted into protective cover 120, air is suctioned out of protective cover 120, causing protective cover 120 to shrink around thermoplastic preform 110. After suctioning, protective cover 120 is sealed (e.g., around the suction port) to maintain a vacuum (or negative pressure) within the interior of protective cover 120. The pressure differential between the inside and outside of protective cover 120 determines an amount of force applied to thermoplastic preform 110. According to implementations described herein, the amount of vacuum pressure inside protective cover 120 required to ensure adequate surface contact between protective cover 120 and thermoplastic preform 110 may be up to 28 Hg (millimeters of mercury), depending on the thickness and pliability of bagging film 322, among other factors. As described below, in other implementations, assembly 100 may be provided with no vacuum pressure inside protective cover 120.

According to another implementation, assembly 100 may be formed without a vacuum seal. For example, a slightly tacky adhesive (e.g., adhesive 324) may be used on all (or substantially all) of the interior surface of top film sheet 432 and/or bottom film sheet 434 that form protective cover 120. Thus, top film sheet 432 and bottom film sheet 434 will both adhere to thermoplastic preform 110 and adhere to each other (e.g., along the outer edges of protective cover 120) to form an airtight/watertight seal around thermoplastic preform 110. In another implementation, top film sheet 432 and bottom film sheet 434 may be single sheet folded into a top and bottom section. Use of adhesive 324 on substantially all of the interior surfaces of top film sheet 432 and bottom film sheet 434 may minimize air entrapment within protective cover 120 and ensure there is some surface contact between protective cover 120 and thermoplastic preform 110, particularly when thermoplastic preform 110 is a substantially flat preform.

In still another implementation, protective cover 120 may be formed using stretch wrap or plastic wrap (e.g., an elastic linear low-density polyethylene (LLDPE)). The wrap may be used to encase thermoplastic preform 110 and may rely on the elastic properties and cling (e.g., static electric charge) of the wrap material to ensure there is surface contact between protective cover 120 and thermoplastic preform 110. In one embodiment, edges of protective cover 120 may be heat sealed after the wrap material is applied over thermoplastic preform 110. In another embodiment, the cling between top film sheet 432 and bottom film sheet 434 may form an adequate seal around the edges of protective cover 120.

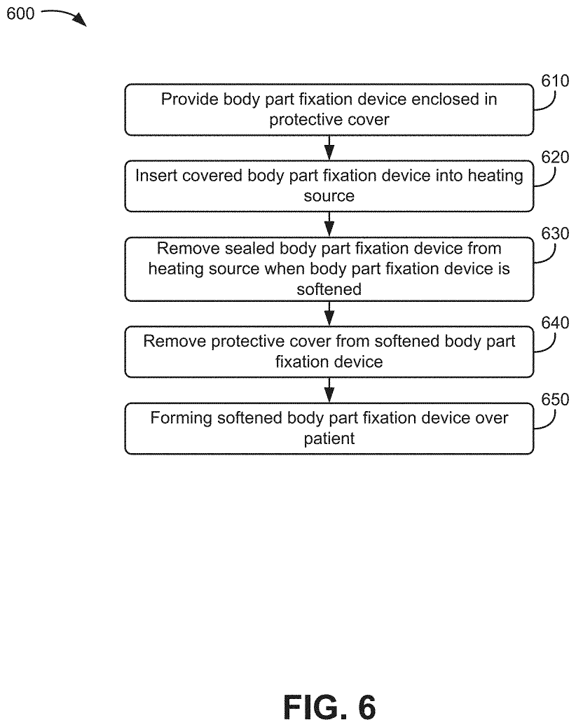

FIG. 5 is a schematic illustrating placement of assembly 100 into an external heating source 510 according to an implementation. As shown in FIG. 5, assembly 100 may be inserted into heating source 510, such as a water bath (as shown) or heating oven. Prior removal of air (using a vacuum seal) from inside protective cover 120 reduces buoyancy of assembly 100 and prevents assembly 100 from floating on the top of the water when heating source 510 is a water bath. Prior removal of air also limits insulating properties of air around thermoplastic preform 110 when heating source 510 is either water or a dry oven. Particularly, when heating source 510 is a water bath, the protective cover 120 should be in contact with as much surface area of thermoplastic preform 110 as possible to obtain good heat transfer from the heated water to the thermoplastic substrate of thermoplastic preform 110. When heating source 510 is a dry oven, air should not be present within protective cover 120 so as to not insulate thermoplastic preform 110 from the warmer air of the dry oven. In both cases, the absence of air increases the uniformity of the heating to the thermoplastic substrate in thermoplastic preform 110. The protective covering also seals thermoplastic preform 110 off from any water or airborne bacteria that may be present in heating source 510 (e.g., a water bath or a heating oven).

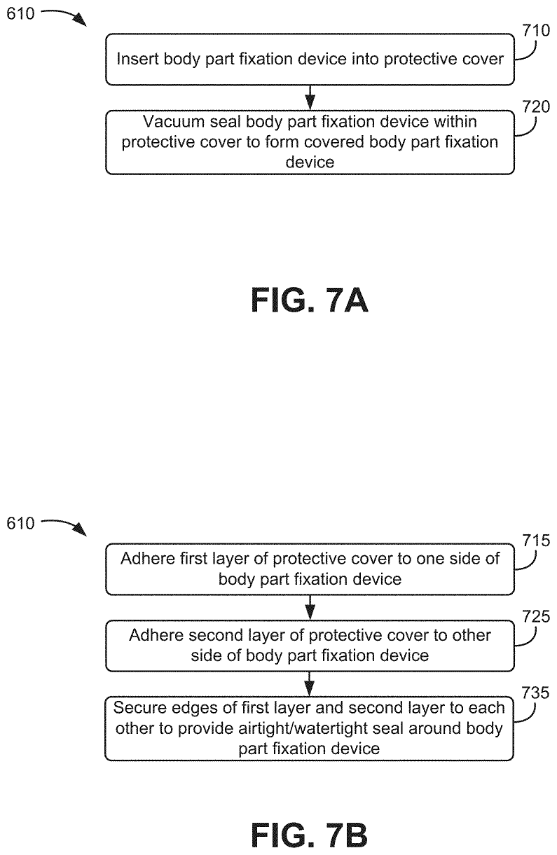

FIGS. 6, 7A, and 7B are flow diagrams of a process 600 for using a thermoplastic preform assembly, according to an implementation described herein. Process 600 may include providing a body part fixation device enclosed in a protective cover (block 610), and inserting the covered body part fixation device into a heating source (block 620). For example, assemblies 100, each including thermoplastic preform 110 within protective cover 120, may be provided in a box or stack. One of assemblies 100 may be removed from the box by a technician when needed and placed in heating source 510 (e.g., either a water bath or dry oven) to be heated to an appropriate softening temperature.

Process 600 may further include removing the covered body part fixation device from heating source when body part fixation device is softened (block 630), and removing the protective cover from softened body part fixation device (block 640). For example, after a specified minimum heating time (e.g., about 5-15 minutes for a dry oven, or about 2-5 minutes for a water bath), the technician may remove assembly 100 from heating source 510. The technician may, for example, simply grasp an edge of protective cover 120. If protective cover 120 is equipped with a seam that can be manually peeled apart, the technician may pull apart top film sheet 432 and bottom film sheet 434 to access thermoplastic preform 110. If protective cover 120 is equipped with a notch 326, the technician may break the vacuum seal by manually pulling at notch 326 to break the vacuum seal and tear an opening in bagging film 322 to access thermoplastic preform 110. In another implementation, scissors or another device may be used to cut bagging film 322.

Process 600 may also include forming the softened body part fixation device over patient (block 650). For example, as illustrated in FIG. 8, once protective cover 120 is removed from the softened thermoplastic preform 110, thermoplastic preform 110 may be applied over a body part of the patient (e.g., within the maximum molding time for thermoplastic preform 110, such as within 3-4 minutes after removal from heating source 510). Thermoplastic preform 110 formed to the shape of the patient, and frame 220 may be secured to a mounting platform 810 or headrest using, for example, pins 820, clips or other fasteners. By removing protective cover 120 after heating and just before applying to the patient, protective cover 120 provides a barrier for thermoplastic preform 110 against airborne or waterborne bacteria that may be present especially in heating source 510.

Referring to FIG. 7A, in one implementation, process block 610 may include inserting a body part fixation device into a protective cover (block 710), and vacuum sealing the body part fixation device within the protective cover to form a covered body part fixation device (block 720). For example, as described in connection with FIGS. 3-4B, protective cover 120 may be formed over thermoplastic preform 110 using a top film sheet 432 and a bottom film sheet 434. Alternatively, protective cover 120 may be supplied as a pouch with an opening 436 into which thermoplastic preform 110 may be inserted before applying suction and sealing along opening 436.

Referring to FIG. 7B, in another implementation, process block 610 may include adhering a first layer of a protective cover to one side of a body part fixation device (block 715), adhering a second layer of a protective cover to another side of the body part fixation device (block 725), and securing edges of the first layer and the second layer to each other to provide an airtight and watertight seal around the body part fixation device (block 735). For example, as described in connection with FIGS. 3 and 4A, protective cover 120 may be formed over thermoplastic preform 110 using a top film sheet 432 and a bottom film sheet 434 with adhesive 324 on substantially all of their interior surfaces. Thus, top film sheet 432 and bottom film sheet 434 will both adhere to thermoplastic preform 110 and adhere to each other (e.g., along the outer edges of protective cover 120) to form an airtight/watertight seal around thermoplastic preform 110.

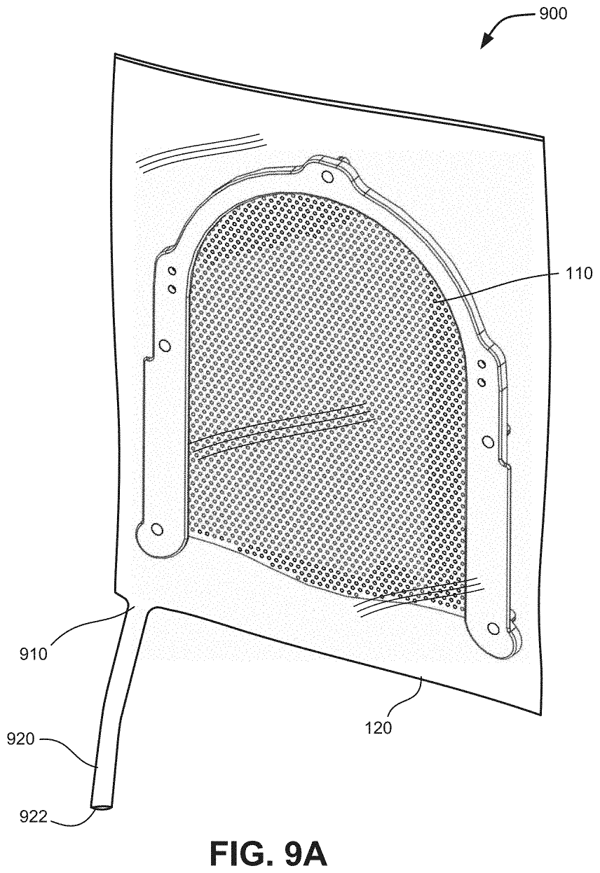

FIGS. 9A and 9B are illustrations of a thermoplastic preform assembly 900 including a thermoplastic preform 110 within protective cover 120, according to another implementation described herein. Similar to embodiments described above protective cover 120 is applied over the thermoplastic preform 110 (shown herein as a mask for simplicity, but other preform shapes may be used) to protect a patient from possible exposure to unwanted bacteria which may otherwise attach to preform 110 during the heating process. According to the embodiments of FIGS. 9A and 9B, protective cover 120 may include an air vent 910. Air vent 910 may be connected to or integral with an optional extension tube 920. Generally, air vent 910 (with or without extension tube 920) may allow air from inside protective cover 120 to be forced out by water pressure from a heating source 510 (e.g., water of the water bath).

In one implementation, air vent 910 may include an opening in protective cover 120 to allow air from inside protective cover 120 to pass out. Particularly, external pressure from outside protective cover 120 may force air from inside protective cover 120 through air vent 910. In another implementation, air vent 910 may use a one-way valve, such that air can be forced out of protective cover 120 but cannot flow back in.

Extension tube 920 may be formed from a flexible material or rigid material that will not deform at temperatures of the heating source. In one implementation, as shown in FIG. 9A, air vent 910 and tube 920 may be integrally formed with protective cover 120 and from the same material. In another implementation, air vent 910 and extension tube 920 may be a separate piece attached to protective cover 120. For example, extension tube may be removably attached to air vent 910 (e.g., via an interference fit, threaded connection, etc.).

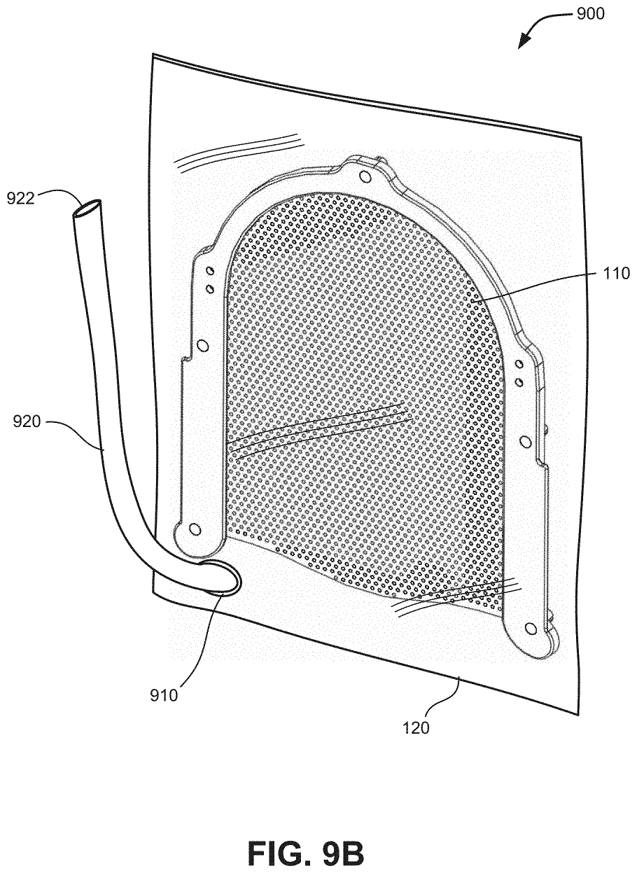

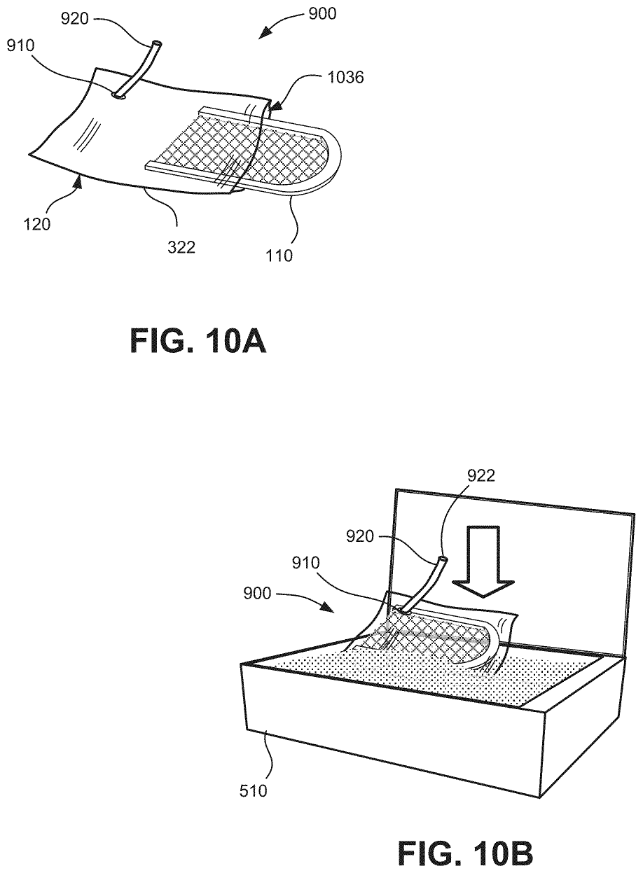

FIG. 10A is an assembly view of the thermoplastic preform assembly 900 according to an embodiment. As shown in FIG. 10A, protective cover 120 may be supplied with bagging film 322 forming a pouch with an opening 1036 into which thermoplastic preform 110 may be inserted. Protective cover 120 may be sealed along opening 1036 with thermoplastic preform 110 enclosed therein.

FIG. 10B is a schematic illustrating placement of assembly 900 into an external heating source 510, according to an implementation. As shown in FIG. 10B, assembly 900 may be inserted into heating source 510 in the form of a water bath. As assembly 900 is inserted, the opening 922 of extension tube 920 may remain above the water level in heating source 510. Pressure of the water in heating source 510 may force air out through air vent 910 and extension tube 920. Removal of air from protective cover 120 allows protective cover 120 to be in good contact with thermoplastic preform 110 and obtain good heat transfer from the heated water to the thermoplastic substrate of thermoplastic preform 110. With opening 922 extended above the water level, protective cover 120 also protects thermoplastic preform 110 off from waterborne bacteria that may be present in heating source 510. After heating, assembly 900 may be used as described above, for example, in connection with FIG. 6.

In implementations described herein, an assembly for a thermoplastic preform for a body part is provided. The thermoplastic preform is deformable when heated to a softening temperature by an external heating source and hardens when cooled below the softening temperature. The assembly includes the thermoplastic preform and a protective cover surrounding the thermoplastic preform. The thermoplastic preform is vacuum sealed within the protective cover. The protective cover is heat resistant up to a temperature that exceeds the softening temperature for the thermoplastic preform. The protective cover transfers heat from the external heating source to the thermoplastic preform and prevents airborne and waterborne bacteria from contacting the thermoplastic preform.

In contrast with current preforms for thermoplastic retention devices, the protective cover 120 of assembly 100 provides a more robust structure that reduces sagging or deformation of thermoplastic preform 110 when transferring assembly 100 from heat source (e.g., heat source 100) to a patient. Additionally, protective cover 120 slows down the cooling process of thermoplastic preform 110 (since there is no evaporation off the surface of the cooling thermoplastic preform 110), which allows more time for technicians to form thermoplastic preform 110 over a patient. Use of protective cover 120 may also allow thermoplastic preform 110 to be provided in a sterilized condition, by sterilizing thermoplastic preform 110 (e.g., via radiation sterilization processes) prior to or after vacuum sealing in protective cover 120. Furthermore, assembly 100 allows technicians to use the more efficient water bath heating for thermoplastic preform 110 without having to dry thermoplastic preform 110 or apply a wet thermoplastic preform 110 to a patient.

The foregoing description of exemplary implementations provides illustration and description, but is not intended to be exhaustive or to limit the embodiments described herein to the precise form disclosed. Modifications and variations are possible in light of the above teachings or may be acquired from practice of the embodiments. For example, while a series of blocks have been described with regard to FIGS. 6, 7A and 7B, the order of the blocks may be modified in other embodiments. Further, non-dependent blocks may be performed in parallel.

Although the invention has been described in detail above, it is expressly understood that it will be apparent to persons skilled in the relevant art that the invention may be modified without departing from the spirit of the invention. Various changes of form, design, or arrangement may be made to the invention without departing from the scope of the invention. Furthermore, different features illustrated separately above may be combined in a single embodiment. Therefore, the above-mentioned description is to be considered exemplary, rather than limiting, and the true scope of the invention is that defined in the following claims.

Use of ordinal terms such as “first,” “second,” “third,” etc., in the claims to modify a claim element does not by itself connote any priority, precedence, or order of one claim element over another, the temporal order in which acts of a method are performed, the temporal order in which instructions executed by a device are performed, etc., but are used merely as labels to distinguish one claim element having a certain name from another element having a same name (but for use of the ordinal term) to distinguish the claim elements.

No element, act, or instruction used in the description of the present application should be construed as critical or essential to the invention unless explicitly described as such. Also, as used herein, the article “a” is intended to include one or more items. Further, the phrase “based on” is intended to mean “based, at least in part, on” unless explicitly stated otherwise.

What is claimed is:

1. A system, comprising:
a thermoplastic preform for a body part, wherein the thermoplastic preform is deformable when heated to a softening temperature by an external heating source and hardens when cooled below the softening temperature; and
a protective cover surrounding the thermoplastic preform,
wherein the thermoplastic preform is vacuum sealed within the protective cover,
wherein the protective cover is heat resistant up to a temperature that exceeds the softening temperature for the thermoplastic preform,
wherein the protective cover transfers heat from the external heating source to the thermoplastic preform, and
wherein the protective cover prevents airborne and waterborne bacteria from contacting the thermoplastic preform.

2. The system of claim 1, wherein the thermoplastic preform further comprises a frame made from a different material than a material of the thermoplastic preform.

3. The system of claim 2, wherein the frame is not deformable when heated to the softening temperature.

4. The system of claim 2, wherein the frame includes one or more integrated fasteners to secure the frame to a mounting platform.

5. The system of claim 2, wherein the frame includes mounting holes for pins, and wherein the pins are not included within the protective cover.

6. The system of claim 1, wherein the protective cover is manually removable from the thermoplastic preform after the thermoplastic preform is heated to the softening temperature.

7. The system of claim 6, wherein the protective cover includes one or more layers that are manually peeled apart to break the vacuum seal.

8. The system of claim 6, wherein the protective cover includes one or more tabs that can be manually torn to break the vacuum seal.

9. The system of claim 1, wherein the external heating source includes one of a water bath or a dry oven.

10. The system of claim 1, wherein the protective cover is a material made from one or more layers of nylon, polyethylene, ethylene-vinyl alcohol, polyamide, or polyethylene terephthalate.

11. The system of claim 1, wherein the amount of vacuum pressure inside the vacuum sealed protective cover is up to 28 Hg.

12. A method for forming a thermoplastic preform over a patient, the method comprising:
selecting a thermoplastic preform assembly, wherein the thermoplastic preform assembly includes a thermoplastic preform enclosed in a sealed protective cover;
placing the thermoplastic preform assembly in a heating source until the thermoplastic preform reaches a softening temperature;
removing the thermoplastic preform assembly from the heating source;
removing the thermoplastic preform from the sealed protective cover after removing the thermoplastic preform assembly from the heating source; and
forming the thermoplastic preform on a body part of a patient.

13. The method of claim 12, wherein placing the thermoplastic preform assembly in a heating source comprises:
placing the thermoplastic preform assembly into a water bath with a water temperature exceeding 60 degrees Celsius.

14. The method of claim 12, wherein placing the thermoplastic preform assembly in a heating source comprises:
placing the thermoplastic preform assembly into a dry oven with an air temperature exceeding 60 degrees C.

15. The method of claim 12, wherein the sealed protective cover includes a top film sheet and a bottom film sheet, and wherein removing the thermoplastic preform from the sealed protective cover comprises:
manually pulling apart at least a portion of the top film sheet from the bottom film sheet to access the thermoplastic preform.

16. The method of claim 12, wherein the sealed protective cover includes a notch, and wherein removing the thermoplastic preform from the sealed protective cover comprises:
manually pulling at the notch to tear an opening in the sealed protective cover and access thermoplastic preform.

17. A system, comprising:
a thermoplastic preform for a body part, wherein the thermoplastic preform is deformable when heated to a softening temperature by an external heating source and hardens when cooled below the softening temperature; and
a protective cover surrounding the thermoplastic preform,
wherein the protective cover contacts the thermoplastic preform,
wherein the protective cover is heat resistant up to a temperature that exceeds the softening temperature for the thermoplastic preform,
wherein the protective cover transfers heat from the external heating source to the thermoplastic preform; and
wherein the protective cover prevents airborne and waterborne bacteria from contacting the thermoplastic preform.

18. The system of claim 17, wherein the protective cover is manually removable from the thermoplastic preform after the thermoplastic preform is heated to the softening temperature.

19. The system of claim 17, wherein the protective cover includes an adhesive along substantially all of an interior surface of the protective cover 120 such that the protective cover adheres to the thermoplastic preform and adheres to itself to form a seal around the thermoplastic preform.

20. The system of claim 17, wherein the protective over includes an air vent that allows air from inside the protective cover to be forced out by water pressure from a heating source.
